SEQUENCE LISTING (20 sequences)

SEQ ID NO: 1 - DNA, 246 bp, synthetic construct
  misc_feature 1..246: note = atuA 1.0
  source 1..246: mol_type = other DNA
aataggcgtatcacgaggccctttcgtcttcacctcgaggtgtgctcatccatgctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctctctagttgcggccgcaaaatggatggcaaatatgactaaa

SEQ ID NO: 2 - DNA, 283 bp, synthetic construct
  misc_feature 1..283: note = atuA 2.0
  source 1..283: mol_type = other DNA
tcacgaggccctttcgtcttcacctcgagggggagaccagaaacaaaaaaaggggagcggtttcccgctccccttcaataattggttaatagcgtgcaccgctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctcatgactaaaaaaatttcattcattattaac

SEQ ID NO: 3 - DNA, 906 bp, synthetic construct
  misc_feature 1..906: note = atuA-wt 3.0
  source 1..906: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatgctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctatgactaaaaaaatttcattcattattaac

SEQ ID NO: 4 - DNA, 1133 bp, synthetic construct
  misc_feature 1..1133: note = atuA-L 3.0
  source 1..1133: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatggcgcagatgctccaggcagtgcgagagagaaacacaaggcaacggttatcccgggacatcgatttttcggcagtcgacggacgggtgttataaagacaccccgaaaattcttgagcccgccgaagcggtgatcaggcgctgaagtggcctgtttcaagggtttttggaccaaaacgaaaaaaggcccccctttcgggaggcctcttttctggaatttggtaccgaggctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctatgactaaaaaaatttcattcattattaac

SEQ ID NO: 5 - DNA, 1133 bp, synthetic construct
  misc_feature 1..1133: note = atuA-M 3.0
  source 1..1133: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatatcgtgagcttccttacacacggaaagccctaacgatagatggtcctgagcgaaacgcatcagggtgtcggcgtctcgggacccgacctagctttcctgtaggaactatctcttagcgacgtagggggaagtacaattcagaacaattgttcagaattgtgcgatcggaccaaaacgaaaaaaggcccccctttcgggaggcctcttttctggaatttggtaccgaggctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctatgactaaaaaaatttcattcattattaac

SEQ ID NO: 6 - DNA, 1133 bp, synthetic construct
  misc_feature 1..1133: note = atuA-H 3.0
  source 1..1133: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatatggggtggcccctgattgttgttgtttgatcagcttggataattggtaataccaatttacaatcgctgtgggctagattaagagccttggcaggggtgtgtcaatttgccgctcgtcaaactttggtctgagagtggctgtcgagtgttctgaacgggtaaatcgggaccaaaacgaaaaaaggcccccctttcgggaggcctcttttctggaatttggtaccgaggctgctctccttcacttgtgctgacatcttgggcggcaatttaggccgtggccgccacccaagcaagcgcttgggtaaaacttgtgcgcacagtttacaaaccaagcgcttgcttggtagtctcgacaccagcccctggaggacggatttctatgactaaaaaaatttcattcattattaac

SEQ ID NO: 7 - DNA, 80 bp, synthetic construct
  misc_feature 1..80: note = atuR 1.0
  source 1..80: mol_type = other DNA
aataggcgtatcacgaggccctttcgtcttcacctcgaggtctagttgcggccgcaaaatggatggcaaatatgactaaa

SEQ ID NO: 8 - DNA, 283 bp, synthetic construct
  misc_feature 1..283: note = atuR 2.0
  source 1..283: mol_type = other DNA
tcacgaggccctttcgtcttcacctcgagggggagaccagaaacaaaaaaaggggagcggtttcccgctccccttcaataattggttaatagcgtgcaccgagaaatccgtcctccaggggctggtgtcgagactaccaagcaagcgcttggtttgtaaactgtgcgcacaagttttacccaagcgcttgcttgggtggcggccacggcctaaattgccgcccaagatgtcagcacaagtgaaggagagcagcatgactaaaaaaatttcattcattattaac

SEQ ID NO: 9 - DNA, 907 bp, synthetic construct
  misc_feature 1..907: note = atuR-wt 3.0
  source 1..907: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatgagaaatccgtcctccaggggctggtgtcgagactaccaagcaagcgcttggtttgtaaactgtgcgcacaagttttacccaagcgcttgcttgggtggcggccacggcctaaattgccgcccaagatgtcagcacaagtgaaggagagcagcatgactaaaaaaatttcattcattattaac

SEQ ID NO: 10 - DNA, 1134 bp, synthetic construct
  misc_feature 1..1134: note = atuR-L 3.0
  source 1..1134: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatggcgcagatgctccaggcagtgcgagagagaaacacaaggcaacggttatcccgggacatcgatttttcggcagtcgacggacgggtgttataaagacaccccgaaaattcttgagcccgccgaagcggtgatcaggcgctgaagtggcctgtttcaagggtttttggaccaaaacgaaaaaaggcccccctttcgggaggcctcttttctggaatttggtaccgaggagaaatccgtcctccaggggctggtgtcgagactaccaagcaagcgcttggtttgtaaactgtgcgcacaagttttacccaagcgcttgcttgggtggcggccacggcctaaattgccgcccaagatgtcagcacaagtgaaggagagcagcatgactaaaaaaatttcattcattattaac

SEQ ID NO: 11 - DNA, 1134 bp, synthetic construct
  misc_feature 1..1134: note = atuR-M 3.0
  source 1..1134: mol_type = other DNA
tcacgaggccctttcgtcttcacctcgagggggagaccagaaacaaaaaaaggggagcggtttcccgctccccttcaataattggttaatagcgtgcacctcaatccgcctttaacaccatcagcagggcctcttcggccaattcttcaagggtcaggctgccctgggtacggaaccaggtagtggtccaggacaacgcgcccgtcaggaaacgccgagtaataaacacgtcaccgcggatatacccggcggtcttggcctcacccaatacctgcaaccagatatcttcataaacgtcgcgcaaggccagcacctgggcctggccctcggccgacagcgaacgccattcgtagaccagcaccgccatggcctcgccgctgccgcccatgatcgactgcaactcgcagcgaatcagcgccagcacacgctcgcgtacagtgctcgcctcttccagcgcggcgcgcatcattgcggtgttgtagtggatggtttcttccatcaccgcccgcagaatctcgtccttgctcttgaaatgatgaaagatgctgcccgactgtatgcccacggcgctggcaaggtcacgcacggtagtgcgctcaaaacccttgttgcgaaacaggtgagccgcggtttgcagcaatttgccccgggcgctctcggggtcggtcaatcggccaccggcgaccatggcgcgcatcacccccagggctttgtgctcatccatatggggtggcccctgattgttgttgtttgatcagcttggataattggtaataccaatttacaatcgctgtgggctagattaagagccttggcaggggtgtgtcaatttgccgctcgtcaaactttggtctgagagtggctgtcgagtgttctgaacgggtaaatcgggaccaaaacgaaaaaaggcccccctttcgggaggcctcttttctggaatttggtaccgaggagaaatccgtcctccaggggctggtgtcgagactaccaagcaagcgcttggtttgtaaactgtgcgcacaagttttacccaagcgcttgcttgggtggcggccacggcctaaattgccgcccaagatgtcagcacaagtgaaggagagcagcatgactaaaaaaatttcattcattattaac

SEQ ID NO: 12 - DNA, 1134 bp, synthetic construct; identical to SEQ ID NO: 11
  misc_feature 1..1134: note = atuL-H 3.0
  source 1..1134: mol_type = other DNA

SEQ ID NO: 13 - DNA, 245 bp, synthetic construct
  misc_feature 1..245: note = marR 1.0
  source 1..245: mol_type = other DNA
aataggcgtatcacgaggccctttcgtcttcacctcgaggagcaagcccctcccacattcgcccggcggtgtggccgctgaatcgctccatccatcgagaaattgtttcaaaacttgacgaagactcgcccctccaccatattaatagttagcaaactaactgtatgcgaactatccagtgtcccaatccctcgaacaacttcagtctagttgcggccgcaaaatggatggcaaatatgactaaa

SEQ ID NO: 14 - DNA, 260 bp, synthetic construct
  misc_feature 1..260: note = marR 2.0
  source 1..260: mol_type = other DNA
tcacgaggccctttcgtcttcacctcgagggtaaatgcatctgtggctggcccatcgcaatcgggagcaagcccctcccacattcgcccggcggtgtggccgctgaatcgctccatccatcgagaaattgtttcaaaacttgacgaagactcgcccctccaccatattaatagttagcaaactaactgtatgcgaactatccagtgtcccaatccctcgaacaacttcagatgactaaaaaaatttcattcattattaac

SEQ ID NO: 15 - DNA, 977 bp, synthetic construct
  misc_feature 1..977: note = marR-L 3.0
  source 1..977: mol_type = other DNA
tcacgaggccctttcgtcttcacctcgagggggagaccagaaacaaaaaaaggggagcggtttcccgctccccttcaataattggttaatagcgtgcacctcaaggcgccacagcctcggccgcttcgaaggcgcgcagcacgcgcagtgcggcttccaggtcggtgggcgcgatatccacaaggacttccttgcgcaggcgaaccagttgggcttctaccgcctgcaccagttcacggccgcgctcggtgaggctcagggccttggcgcgacggtcgtggatgtcttcggtgcggcatacgtagccgtcgccgcacagccgatcgagcaggcgcaccagtgacgggctctccatgccggaggcctgggctactttgacctggtgcacaccgtcacccaggcggccgatcatcaacaacggcacggcgcaggcttcggagatgccatagctcaccagcgtggtctggcagatcttgcgccagttacgggcgcccaccaccatgctgctgctcaggttcatggcgcagatgctccaggcagtgcgagagagaaacacaaggcaacggttatcccgggacatcgatttttcggcagtcgacggacgggtgttataaagacaccccgaaaattcttgagcccgccgaagcggtgatcaggcgctgaagtggcctgtttcaagggtttttggaccaaaacgaaaaaaggcccccctttcgggaggcctcttttctggaatttggtaccgaggtaaatgcatctgtggctggcccatcgcaatcgggagcaagcccctcccacattcgcccggcggtgtggccgctgaatcgctccatccatcgagaaattgtttcaaaacttgacgaagactcgcccctccaccatattaatagttagcaaactaactgtatgcgaactatccagtgtcccaatccctcgaacaacttcagatgactaaaaaaatttcattcattattaac

SEQ ID NO: 16 - DNA, 977 bp, synthetic construct
  misc_feature 1..977: note = marR-M 3.0
  source 1..977: mol_type = other DNA
tcacgaggccctttcgtcttcacctcgagggggagaccagaaacaaaaaaaggggagcggtttcccgctccccttcaataattggttaatagcgtgcacctcaaggcgccacagcctcggccgcttcgaaggcgcgcagcacgcgcagtgcggcttccaggtcggtgggcgcgatatccacaaggacttccttgcgcaggcgaaccagttgggcttctaccgcctgcaccagttcacggccgcgctcggtgaggctcagggccttggcgcgacggtcgtggatgtcttcggtgcggcatacgtagccgtcgccgcacagccgatcgagcaggcgcaccagtgacgggctctccatgccggaggcctgggctactttgacctggtgcacaccgtcacccaggcggccgatcatcaacaacggcacggcgcaggcttcggagatgccatagctcaccagcgtggtctggcagatcttgcgccagttacgggcgcccaccaccatgctgctgctcaggttcatatcgtgagcttccttacacacggaaagccctaacgatagatggtcctgagcgaaacgcatcagggtgtcggcgtctcgggacccgacctagctttcctgtaggaactatctcttagcgacgtagggggaagtacaattcagaacaattgttcagaattgtgcgatcggaccaaaacgaaaaaaggcccccctttcgggaggcctcttttctggaatttggtaccgaggtaaatgcatctgtggctggcccatcgcaatcgggagcaagcccctcccacattcgcccggcggtgtggccgctgaatcgctccatccatcgagaaattgtttcaaaacttgacgaagactcgcccctccaccatattaatagttagcaaactaactgtatgcgaactatccagtgtcccaatccctcgaacaacttcagatgactaaaaaaatttcattcattattaac

SEQ ID NO: 17 - DNA, 977 bp, synthetic construct
  misc_feature 1..977: note = marR-H 3.0
  source 1..977: mol_type = other DNA
tcacgaggccctttcgtcttcacctcgagggggagaccagaaacaaaaaaaggggagcggtttcccgctccccttcaataattggttaatagcgtgcacctcaaggcgccacagcctcggccgcttcgaaggcgcgcagcacgcgcagtgcggcttccaggtcggtgggcgcgatatccacaaggacttccttgcgcaggcgaaccagttgggcttctaccgcctgcaccagttcacggccgcgctcggtgaggctcagggccttggcgcgacggtcgtggatgtcttcggtgcggcatacgtagccgtcgccgcacagccgatcgagcaggcgcaccagtgacgggctctccatgccggaggcctgggctactttgacctggtgcacaccgtcacccaggcggccgatcatcaacaacggcacggcgcaggcttcggagatgccatagctcaccagcgtggtctggcagatcttgcgccagttacgggcgcccaccaccatgctgctgctcaggttcatatggggtggcccctgattgttgttgtttgatcagcttggataattggtaataccaatttacaatcgctgtgggctagattaagagccttggcaggggtgtgtcaatttgccgctcgtcaaactttggtctgagagtggctgtcgagtgttctgaacgggtaaatcgggaccaaaacgaaaaaaggcccccctttcgggaggcctcttttctggaatttggtaccgaggtaaatgcatctgtggctggcccatcgcaatcgggagcaagcccctcccacattcgcccggcggtgtggccgctgaatcgctccatccatcgagaaattgtttcaaaacttgacgaagactcgcccctccaccatattaatagttagcaaactaactgtatgcgaactatccagtgtcccaatccctcgaacaacttcagatgactaaaaaaatttcattcattattaac

SEQ ID NO: 18 - DNA, 246 bp, synthetic construct
  misc_feature 1..246: note = 3680 1.0
  source 1..246: mol_type = other DNA
aataggcgtatcacgaggccctttcgtcttcacctcgaggcaccccggtgcaaaaccgcgtgcttgagaggctcgatctagagcctttgcagggaacaatcaattctgttgattattaccattgtgtttatgaacttttatatcgacttatcaagagtagcattagccctgtacccactttatcgccctcccaaggagcaaccatcttctagttgcggccgcaaaatggatggcaaatatgactaa

SEQ ID NO: 19 - DNA, 417 bp, synthetic construct
  misc_feature 1..417: note = 3680 2.0
  source 1..417: mol_type = other DNA
tcacgaggccctttcgtcttcacctcgaggtgcacagggctcctgaatacgactggctgggcaacagcctttgacacagcttatagaactacgactggcaaagtgccggagggtttaacccaacggcggatttagccgaaaaaaacgattttcctgatcaagcagacagatttcatcatcgacagcgcaccattgttaccccggttcgtgtcgaaaatacccaccccggtgcaaaaccgcgtgcttgagaggctcgatctagagcctttgcagggaacaatcaattctgttgattattaccattgtgtttatgaacttttatatcgacttatcaagagtagcattagccctgtacccactttatcgccctcccaaggagcaaccatcatgactaaaaaaatttcattcattattaac

SEQ ID NO: 20 - DNA, 300 bp, synthetic construct
  misc_feature 1..300: note = 207452.0
  source 1..300: mol_type = other DNA
tcacgaggccctttcgtcttcacctcgaggcttggcgcactcgttcgcaggccgcaagccgatcgccctctaacgcccattgcagccccaatgtgggagctggcttgcctgcgatagcatcaccacggtgtaactgaaaaaccgaggcgcttgcatcgcgggcaagcccggctcccacattggttttgtggtgtcggtgaaaatcgtgtacagcttgaaaaccaagcaagcgcttggtaagttcgctccatctcgccatggagcttgccgatgactaaaaaaatttcattcattattaac

(19) United States
(12) Patent Application Publication
(10) Pub. No.: US 2024/0376514 A1
(43) Pub. Date: Nov. 14, 2024

(54) BACTERIAL BIOSENSORS FOR MONITORING AND DETECTION OF NAPHTHENIC ACIDS IN THE ENVIRONMENT

(71) Applicant: THE GOVERNORS OF ATHABASCA UNIVERSITY, ATHABASCA (CA)

(72) Inventor: William Shawn LEWENZA, Squamish (CA)

(21) Appl. No.: 18/691,819

(22) PCT Filed: Sep. 16, 2022
(86) PCT No.: PCT/CA2022/051379

§ 371 Date: Mar. 13, 2024

Related U.S. Application Data
(60) Provisional application No. 63/245,435, filed on Sep. 17, 2021.

(51) Int. Cl. C12Q 1/02; C12N 1/20; C12R 1/39; G01N 33/18

(52) U.S. Cl. CPC C12Q 1/02; C12N 1/20; G01N 33/1826; C12N 2510/00; C12R 2001/39

(57) ABSTRACT

A bacterial biosensor comprises a modified Pseudomonas species carrying a plasmid or chromosomally encoding a genetic circuit having a reporter gene fused with a promoter responsive to the presence of naphthenic acids. The genetic circuit is expressed proportionally in the presence of naphthenic acids in the environment. Methods of detecting the presence of naphthenic acids in an environment or monitoring degradation of NAs by a bacterial strain using the bacterial biosensor are provided.

### CROSS-REFERENCE TO RELATED APPLICATION

The present application claims the benefit of U.S. Provisional Application No. 63/245,435, filed Sep. 17, 2021, the disclosure of which is incorporated herein by reference in its entirety.

### FIELD OF THE INVENTION

The present invention relates to bacterial biosensors for monitoring and detection of naphthenic acids in the environment.

### BACKGROUND OF THE INVENTION

Oil sands are a combination of bitumen, quartz sand, clay, water, and trace minerals, and can be excavated using surface mining and in situ drilling. Various bitumen extraction processes generally include the steps of preparing an oil sand and water slurry from the mined oil sands, conditioning the oil sand slurry, and subjecting the oil sand slurry to a separation process to recover the bitumen. One bitumen extraction process commonly used in the industry is the “hot water process” which involves feeding the mined oil sand into a rotating tumbler where it is mixed for a prescribed retention time with hot water, steam, caustic, and naturally entrained air to yield a slurry having a temperature typically around 80° C. The bitumen matrix is heated and becomes less viscous. Chunks of oil sand are ablated or disintegrated. The released sand grains and separated bitumen flecks are dispersed in the water. To some extent bitumen flecks coalesce and contact air bubbles and coat them to become acrated bitumen. The conditioned slurry is introduced into a separation vessel to recover the bitumen. The hot water process produces good bitumen recoveries for all grades of oil sand.

However, surface mining and hot water extraction of bitumen have contributed to the accumulation of large volumes of oil sands process-affected water (“OSPW”). OSPW contains water, sand, clay, silt, dissolved ions, heavy metals, unrecovered oil, and organic compounds, some of which are constituents of concern that limit the ability to discharge the impacted water into receiving environments due to their toxicity. Naphthenic acids (“NAs”) are the most toxic compounds in OSPW and are generally present in concentrations between 20-120 mg/L. NAs are a complex mixture of monocyclic, polycyclic, acyclic, and alkyl-substituted carboxylic acids that have demonstrated toxicity against microbes, plankton, plants, fish, and mammals. NAs are naturally present in the bitumen mined for petroleum production, and consist of simple compounds that are easily biodegraded, as well as more recalcitrant compounds with complex structures that are slower to degrade.

OSPW is typically stored on-site in large tailings ponds, with infrastructure in place to minimize leakage and collect seepage water. Untreated OSPW is not released into the environment. The industry has been attempting to develop low greenhouse gas generating technologies to remediate OSPW, and to implement approved environmental monitoring strategies to treat OSPW before discharging the impacted water into natural environments and reclaiming the land. Current analytical chemistry methods exist to determine the concentration of NAs in OSPW. However, such methods may require complex sample extraction and preparation, sophisticated equipment (for example, mass spectrometry), highly trained equipment operators, or challenging analytical chemistry methods to extract, detect and characterize the compounds present in OSPW. Such methods are slow, expensive, and unsuitable for high throughput sample measurements. To date, there is no universally approved standard method for detecting NAs in OSPW.

While bacteria have been widely used for genetic manipulation, they can also be genetically engineered to function as bacterial biosensors capable of sensing or detecting and quantifying numerous analytes including, but not limited to, various aromatic compounds (for example, benzene, toluene, xylene, phenol, and naphthalene), alkanes, solvents, sugars, heavy metals, and antibiotics (van Der Meer and Belkin, 2010, Nature Reviews Microbiology, 8, 511-522). A whole-cell bacterial biosensor consists of genetically engineered bacteria containing a contaminant-inducible gene capable of detecting the presence of an analyte, coupled with a reporter gene capable of producing a measurable output response. The sensitivity for bacterial biosensors is commonly in the parts per million (ppm, mg/L) range, but can be sensitive to parts per billion (ppb, μg/L).

Very little is known about gene expression in bacteria recovered from oil sands tailings ponds. One study has examined the bacterial gene expression responses of E. coli K12 (Zhang et al., 2011, Environ. Sci. and Technol., 45 (5), 1984-1991). However, since it originated from a patient stool sample and has been a lab-adapted strain since its isolation in 1922, E. coli K12 is unsuitable for use in examining responses to compounds in an oil sands deposit, OSPW, or tailings pond. A publicly available library of transcriptional reporters was constructed to the fluorescent GFP protein, and only 75% of all predicted promoters in E. coli were included in the library. Using this pre-constructed library, gene expression responses of E. coli were measured after exposure to a commercial NAs technical mixture which contained predominantly acyclic carboxylic acids and a few cyclic compounds. The mixture thus failed to represent the diverse NAs commonly found in oil sands. Another study has reported an RNA-seq transcriptome approach to identify Pseudomonas genes expressed in the presence of NAs extracted from OSPW (Chegounian et al., 2021, Microorganisms, 9 (10): 2124). Accordingly, there is a need for bacterial biosensors capable of detecting a wide range of NAs, particularly those in OSPW.

### SUMMARY OF THE INVENTION

The present invention relates to relates to bacterial biosensors for monitoring and detection of naphthenic acids in the environment.

Broadly, in one aspect, the invention comprises a biosensor for detecting the presence of naphthenic acids in an environment, comprising a modified Pseudomonas species carrying a plasmid or chromosomally encoding a genetic circuit comprising a reporter gene fused with a promoter responsive to the presence of naphthenic acids, the genetic circuit being expressed in the presence of naphthenic acids in the environment.

In some embodiments, the modified Pseudomonas species is selected from the Pseudomonas fluorescens complex of species, or Pseudomonas species strain OST1909.

In some embodiments, the reporter gene is selected from luxCDABE, beta-galactosidase, or green fluorescent protein. In some embodiments, the promoter is selected from the promoter IH404_19875, atuA, IH404_01620, marR, IH404_03680, or 3680.

In some embodiments, the genetic circuit comprises a promoter responsive to the presence of naphthenic acids, and a transcriptional repressor protein controlling the promoter.

In another aspect, the invention comprises a genetic circuit comprising an isolated nucleic acid sequence as set forth in any one of SEQ ID NOS: 1-20, or an isolated nucleic acid sequence having at least 80%, at least 85%, at least 90%, at least 95%, at least 96%, at least 97%, at least 98%, or at least 99% sequence identity to any one of SEQ ID NOS: 1-20.

In another aspect, the invention comprises an isolated nucleic acid sequence as set forth in any one of SEQ ID NOS: 1-20, or an isolated nucleic acid sequence having at least 80%, at least 85%, at least 90%, at least 95%, at least 96%, at least 97%, at least 98%, or at least 99% sequence identity to any one of SEQ ID NOS: 1-20.

In another aspect, the invention comprises a method of detecting the presence of naphthenic acids in an environment comprising: providing a sample from the environment suspected of containing naphthenic acids; and contacting the sample with the above biosensor; wherein a detectable signal is induced in the biosensor, the detectable signal being indicative of naphthenic acids in the environment.

In another aspect, the invention comprises a method for detecting the presence of naphthenic acids in an environment comprising: contacting a sample from the environment suspected of containing naphthenic acids with the above biosensor; and detecting color, electrochemical, or light emitted by a gene product that is induced by the presence of naphthenic acids, the intensity of the color or light being indicative of the concentration of naphthenic acids in the environment.

In some embodiments, the environment comprises water from the ground, soil, river, lake, stream, pond, tailings pond, wastewater, or oil sands process-affected water. In some embodiments, the water comprises oil sands process-affected water. In some embodiments, the naphthenic acids in the oil sands process-affected water range in concentration from about 20 mg/L to about 120 mg/L. In some embodiments, the detectable signal comprises color or light emitted by luxCDABE, beta-galactosidase, or green fluorescent protein.

In yet another aspect, the invention comprises a method for monitoring degradation of naphthenic acids by a bacterial strain comprising: growing a bacterial strain in the presence of a known amount of naphthenic acids; and contacting the bacterial strain with the above biosensor; wherein a detectable signal is induced in the biosensor, the detectable signal being indicative of naphthenic acids degraded by the bacterial strain. In some embodiments, the detectable signal comprises color, electrochemical, or light emitted by luxCDABE, beta-galactosidase, or green fluorescent protein.

Additional aspects and advantages of the present invention will be apparent in view of the description, which follows. It should be understood, however, that the detailed description and the specific examples, while indicating preferred embodiments of the invention, are given by way of illustration only, since various changes and modifications within the scope of the invention will become apparent to those skilled in the art.

### BRIEF DESCRIPTION OF THE DRAWINGS

The invention will now be described by way of an exemplary embodiment with reference to the accompanying simplified, diagrammatic, not-to-scale drawings. In the drawings:

FIG. 1 is a schematic diagram showing one embodiment of a pipeline (steps 1-4) of naphthenic acid biosensor assembly and validation.

FIGS. 2A-D are schematic diagrams showing genetic biosensor circuit designs in luxCDABE reporter plasmid pMS402. FIGS. 2A-B show promoters with native RBS cloned into the wild type pMS402 plasmid (version 1.0) and other promoters cloned without the plasmid-encoded RBS (version 2.0). FIG. 2C shows the inclusion of an NA-sensing repressor protein expressed by its constitutive promoter. FIG. 2D shows low, medium or high constitutive promoters controlling the expression of the NA-sensing repressor protein. FIG. 2E shows a key for FIGS. 2A-D.

FIGS. 3A-F are schematic diagrams and graphs showing the genetic organization of promoters that were synthesized and used in biosensor constructs for the detection of NAs (FIGS. 3A-C), and the linear, dose-dependent increase in gene expression by the biosensor in response to increasing concentrations of different mixtures of NAs (FIGS. 3D-F). The range of NA detection is 8-500 mg/L of acyclic NAs (FIG. 3D), 16-500 mg/L of OSPW (FIG. 3E) and 2->128 mg/L of a simple custom mixture of nine compounds (FIG. 3F). The dose-dependent gene expression responses allow these biosensors to provide a semi-quantitative indication of the concentration of NA in a sample.

FIGS. 4A-B are graphs showing detection by the atuA-L biosensor of acyclic NA mixes and individual NA compounds. FIG. 4A is a graph showing the fold gene expression values of four different NA mixes with increasing concentrations. “SIGMA” and “Acros” indicate two sources of primarily acyclic NA mixtures. FIG. 4B is a graph showing the induction (fold gene induction) values of 28 different NAs at a concentration of 50 mg/L.

FIGS. 5A-B are graphs showing detection by the marR-L biosensor of NA extracted from OSPW and individual NAs. FIG. 5A is a graph showing the fold gene expression values of four different NA mixtures with increasing concentrations. FIG. 5B is a graph showing the fold gene expression values of 28 different NAs at a concentration of 50 mg/L.

FIGS. 6A-B are graphs showing detection by the p3680 biosensor of the custom 9xNA mix and individual NA compounds. FIG. 6A is a graph showing the fold gene expression values of four different NA mixtures with increasing concentrations. The 9xNA mix is composed of nine individual commercially available NA compounds. FIG. 6B is a graph showing the fold gene expression values of 28 different NAs at a concentration of 50 mg/L.

FIG. 7 is a graph showing the biosensor responses to naphthenic acids in OSPW samples.

### DETAILED DESCRIPTION OF PREFERRED EMBODIMENTS

Before the present invention is described in further detail, it is to be understood that the invention is not limited to the particular embodiments described, as such may, of course, vary. It is also to be understood that the terminology used herein is for the purpose of describing particular embodiments only, and is not intended to be limiting, since the scope of the present invention will be limited only by the appended claims. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

The present invention relates to bacterial biosensors and methods of using same for monitoring and detection of naphthenic acids in the environment. The invention overcomes the limitations of conventional analytical methods which include high cost, low throughput, complex methods, equipment and training needs, and lack of in-field testing. The invention further relates to a method of recovering bacterial isolates directly from water and applying genome-wide methods to capture the complete bacterial gene expression response and to identify the genes which are induced and highly expressed in response to exposure to various samples of naphthenic acids.

As used herein, the term “bacterial biosensor” refers to a bacterium which is genetically engineered for use as a biosensor which can sense or detect the presence of a certain target analyte of interest and produce a detectable response to quantify the analyte. A hallmark feature of a bacterium is the ability to sense or detect and respond to changing environmental conditions by inducing the expression of relevant genes required for the response.

In some embodiments, the analyte of interest comprises naphthenic acids. As used herein, the term “naphthenic acids (“NAs”) refers to a complex mixture of cyclic and acyclic saturated carboxylic acids that originate by the microbial degradation of petroleum hydrocarbons, and are found in oil sands bitumen, crude oil, petroleum, and coal. The primary hazard of NAs is the toxicity which poses a threat to the environment.

As used herein, the term “environment” refers to the complex of physical, chemical, and biotic factors that act upon an organism or an ecological community and ultimately determine its form and survival. In some embodiments, the term is meant to refer to water, soil, or sediment. In some embodiments, the water may be sourced from the ground, soil, river, lake, stream, pond, tailings pond, wastewater, or oil sands process-affected water.

As used herein, the term “oil sands process-affected water” (“OSPW”) refers to a by-product of bitumen extraction in the surface-mining oil sands industry. OSPW comprises a complex matrix of organic contaminants including NAs, residual bitumen, and polycyclic aromatic hydrocarbons.

In one aspect, the invention comprises a bacterial biosensor for monitoring and detection of NAs in the environment. In some embodiments, the bacterium comprises a whole cell. In some embodiments, the bacterium comprises a Pseudomonas species. In some embodiments, the Pseudomonas species is selected from the Pseudomonas fluorescens complex of species. As used herein, the term “Pseudomonas fluorescens complex” refers to Pseudomonas strains that have been taxonomically assigned to more than fifty different species including, but not limited to, Pseudomonas corrugata, Pseudomonas brassicacearum, Pseudomonas frederiksbergensis, Pseudomonas mandelii, Pseudomonas kribbensis, Pseudomonas koreensis, Pseudomonas mucidolens, Pseudomonas veronii, Pseudomonas antarctica, Pseudomonas azotoformans, Pseudomonas trivialis, Pseudomonas turida, Pseudomonas azotoformans, Pseudomonas poae, Pseudomonas libanensis, Pseudomonas synxantha, and Pseudomonas orientalis.

The choice of a Pseudomonas species for the bacterial biosensor is appropriate since Pseudomonas species are abundant in the environment such as, for example, OPSW and tailings ponds, and are known to be capable of degrading NAs. In some embodiments, the bacterium comprises a Pseudomonas species strain OST1909 which is an isolate recovered from OSPW (Shideler et al., 4 Mar. 2021, Microbiology Resource Announcements).

In some embodiments, the bacterial biosensor comprises a modified Pseudomonas species carrying a plasmid or chromosomally encoding a genetic circuit for detecting NAs in the environment. In some embodiments, the bacterial biosensor comprises a modified Pseudomonas species selected from the Pseudomonas fluorescens complex of species, and carrying a plasmid encoding a genetic circuit for detecting NAs in the environment. In some embodiments, the bacterial biosensor comprises a modified Pseudomonas species strain OST1909 carrying a plasmid encoding a genetic circuit for detecting NAs in the environment.

As used herein, the term “genetic circuit” refers to an assembly of biological parts encoding RNA or protein which enables the bacterial biosensor to respond in the presence of a certain compound of interest.

In some embodiments, the bacterial biosensor expresses the genetic circuit comprising a bacterial promoter that is responsive to the compound and fused to a transcriptional reporter. In some embodiments, the reporter comprises an enzyme which produces a color change or a fluorescence signal. In some embodiments, the enzyme comprises beta-galactosidase which produces a color or electrochemical change. In some embodiments, the enzyme comprises green fluorescent protein which produces a fluorescence signal. In some embodiments, the reporter comprises a luxCDABE reporter capable of bioluminescence. The lux reporter is a suitable reporter of gene expression due to the dynamic range of light production, strong signal-noise ratios, ability to measure real-time luminescence without the addition of substrates, and case of high throughput experiments in 96 to 384-well format plates.

In some embodiments, the genetic circuit comprises a promoter which is fused with the luxCDABE reporter genes, and which is expressed in the presence of NAs.

In some embodiments, the genetic circuit comprises the luxCDABE reporter genes fused with the promoter responsive to the presence of NAs.

In some embodiments, the genetic circuit comprises a promoter which is induced by NAs and a transcriptional repressor protein that controls the NA-inducible promoter. The transcription repressor proteins are also the NA sensing proteins. The transcription repressor proteins reduce expression of the NA genes in the absence of NAs, but no longer repress these genes after sensing NAs.

In some embodiments, the promoter is selected from the promoter IH404_19875, atuA, IH404_01620, marR, IH404_03680, or 3680.

In some embodiments, the genetic circuit comprises an isolated nucleic acid sequence as set forth in any one of SEQ ID NOS: 1-20 (Table 1; regions in bold and the vector flanking sequences required for cloning as described in the Examples). As used herein, the term “isolated” means that a substance or a group of substances is removed from the coexisting materials of its natural state. Nucleic acid sequences having at least 80% homology, more preferably at least 85% homology, more preferably at least 90% homology, more preferably at least 95% homology, or more preferably at least 96%, 97%, 98%, or 99%, homology with any of the nucleic acid sequences described herein are within the scope of this invention. Accordingly, in some embodiments, the genetic circuit comprises a nucleic acid sequence having at least 80%, at least 85%, at least 90%, at least 95%, at least 96%, at least 97%, at least 98%, or at least 99% sequence identity to any one of SEQ ID NOS: 1-20. Methods for isolation of such nucleic acid sequences are well known in the art.

In some embodiments, the invention comprises an isolated nucleic acid having the nucleic acid sequence as set forth in any one of SEQ ID NOS: 1-20, or having at least 80%, at least 85%, at least 90%, at least 95%, at least 96%, at least 97%, at least 98%, or at least 99% sequence identity therewith.

TABLE 1
Synthetic DNA Sequences used in exemplary bacterial biosensors

SEQ ID NO: 1-atuA 1.0
AATAGGCGTATCACGAGGCCCTTTCGTCTTCACCTCGAGGTGTGCTCATCCATGCTGCTCTCC
TTCACTTGTGCTGACATCTTGGGCGGCAATTTAGGCCGTGGCCGCCACCCAAGCAAGCGCT
TGGGTAAAACTTGTGCGCACAGTTTACAAACCAAGCGCTTGCTTGGTAGTCTCGACACCAG
CCCCTGGAGGACGGATTTCTCTCTAGTTGCGGCCGCAAAATGGATGGCAAATATGACTAAA
SEQ ID NO: 2-atuA 2.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCGCTGCTCTCCTTCACTTGTGCTGACATCTTG
GGCGGCAATTTAGGCCGTGGCCGCCACCCAAGCAAGCGCTTGGGTAAAACTTGTGCGCACAGTTT
ACAAACCAAGCGCTTGCTTGGTAGTCTCGACACCAGCCCCTGGAGGACGGATTTCTCATGACTAA
AAAAATTTCATTCATTATTAAC
SEQ ID NO: 3-atuA-wt 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATGCTGCTCTCCTTCACTTGTGCTGACATCTTGGGCGGCAATTTAGGCCGTGGCCGCCA
CCCAAGCAAGCGCTTGGGTAAAACTTGTGCGCACAGTTTACAAACCAAGCGCTTGCTTGGTAGTC
TCGACACCAGCCCCTGGAGGACGGATTTCTATGACTAAAAAAATTTCATTCATTATTAAC
SEQ ID NO: 4-atuA-L 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATGGCGCAGATGCTCCAGGCAGTGCGAGAGAGAAACACAAGGCAACGGTTATCCCGG
GACATCGATTTTTCGGCAGTCGACGGACGGGTGTTATAAAGACACCCCGAAAATTCTTGAGCCCG
CCGAAGCGGTGATCAGGCGCTGAAGTGGCCTGTTTCAAGGGTTTTTGGACCAAAACGAAAAAAG
GCCCCCCTTTCGGGAGGCCTCTTTTCTGGAATTTGGTACCGAGGCTGCTCTCCTTCACTTGTGCTG
ACATCTTGGGCGGCAATTTAGGCCGTGGCCGCCACCCAAGCAAGCGCTTGGGTAAAACTTGTGCG
CACAGTTTACAAACCAAGCGCTTGCTTGGTAGTCTCGACACCAGCCCCTGGAGGACGGATTTCTA
TGACTAAAAAAATTTCATTCATTATTAAC
SEQ ID NO: 5-atuA-M 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATATCGTGAGCTTCCTTACACACGGAAAGCCCTAACGATAGATGGTCCTGAGCGAAAC
GCATCAGGGTGTCGGCGTCTCGGGACCCGACCTAGCTTTCCTGTAGGAACTATCTCTTAGCGACG
TAGGGGGAAGTACAATTCAGAACAATTGTTCAGAATTGTGCGATCGGACCAAAACGAAAAAAGG
CCCCCCTTTCGGGAGGCCTCTTTTCTGGAATTTGGTACCGAGGCTGCTCTCCTTCACTTGTGCTGAC
ATCTTGGGCGGCAATTTAGGCCGTGGCCGCCACCCAAGCAAGCGCTTGGGTAAAACTTGTGCGCA
CAGTTTACAAACCAAGCGCTTGCTTGGTAGTCTCGACACCAGCCCCTGGAGGACGGATTTCTATG
ACTAAAAAAATTTCATTCATTATTAAC
SEQ ID NO: 6-atuA-H 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATATGGGGTGGCCCCTGATTGTTGTTGTTTGATCAGCTTGGATAATTGGTAATACCAAT
TTACAATCGCTGTGGGCTAGATTAAGAGCCTTGGCAGGGGTGTGTCAATTTGCCGCTCGTCAAAC
TTTGGTCTGAGAGTGGCTGTCGAGTGTTCTGAACGGGTAAATCGGGACCAAAACGAAAAAAGGC
CCCCCTTTCGGGAGGCCTCTTTTCTGGAATTTGGTACCGAGGCTGCTCTCCTTCACTTGTGCTGACA
TCTTGGGCGGCAATTTAGGCCGTGGCCGCCACCCAAGCAAGCGCTTGGGTAAAACTTGTGCGCAC
AGTTTACAAACCAAGCGCTTGCTTGGTAGTCTCGACACCAGCCCCTGGAGGACGGATTTCTATGA
CTAAAAAAATTTCATTCATTATTAAC
SEQ ID NO: 7-atuR 1.0
AATAGGCGTATCACGAGGCCCTTTCGTCTTCACCTCGAGGTCTAGTTGCGGCCGCAAAATGGATG
GCAAATATGACTAAA
SEQ ID NO: 8-atuR 2.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCGAGAAATCCGTCCTCCAGGGGCTGGTGTC
GAGACTACCAAGCAAGCGCTTGGTTTGTAAACTGTGCGCACAAGTTTTACCCAAGCGCTTGCTTG
GGTGGCGGCCACGGCCTAAATTGCCGCCCAAGATGTCAGCACAAGTGAAGGAGAGCAGCATGA
CTAAAAAAATTTCATTCATTATTAAC
SEQ ID NO: 9-atuR-wt 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATGAGAAATCCGTCCTCCAGGGGCTGGTGTCGAGACTACCAAGCAAGCGCTTGGTTTG
TAAACTGTGCGCACAAGTTTTACCCAAGCGCTTGCTTGGGTGGCGGCCACGGCCTAAATTGCCGC
CCAAGATGTCAGCACAAGTGAAGGAGAGCAGCATGACTAAAAAAATTTCATTCATTATTAAC
SEQ ID NO: 10-atuR-L 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATGGCGCAGATGCTCCAGGCAGTGCGAGAGAGAAACACAAGGCAACGGTTATCCCGG
GACATCGATTTTTCGGCAGTCGACGGACGGGTGTTATAAAGACACCCCGAAAATTCTTGAGCCCG
CCGAAGCGGTGATCAGGCGCTGAAGTGGCCTGTTTCAAGGGTTTTTGGACCAAAACGAAAAAAG
GCCCCCCTTTCGGGAGGCCTCTTTTCTGGAATTTGGTACCGAGGAGAAATCCGTCCTCCAGGGGC
TGGTGTCGAGACTACCAAGCAAGCGCTTGGTTTGTAAACTGTGCGCACAAGTTTTACCCAAGCGC
TTGCTTGGGTGGCGGCCACGGCCTAAATTGCCGCCCAAGATGTCAGCACAAGTGAAGGAGAGCA
GCATGACTAAAAAAATTTCATTCATTATTAAC
SEQ ID NO: 11-atuR-M 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATATGGGGTGGCCCCTGATTGTTGTTGTTTGATCAGCTTGGATAATTGGTAATACCAAT
TTACAATCGCTGTGGGCTAGATTAAGAGCCTTGGCAGGGGTGTGTCAATTTGCCGCTCGTCAAAC
TTTGGTCTGAGAGTGGCTGTCGAGTGTTCTGAACGGGTAAATCGGGACCAAAACGAAAAAAGGC
CCCCCTTTCGGGAGGCCTCTTTTCTGGAATTTGGTACCGAGGAGAAATCCGTCCTCCAGGGGCTG
GTGTCGAGACTACCAAGCAAGCGCTTGGTTTGTAAACTGTGCGCACAAGTTTTACCCAAGCGCTT
GCTTGGGTGGCGGCCACGGCCTAAATTGCCGCCCAAGATGTCAGCACAAGTGAAGGAGAGCAGC
ATGACTAAAAAAATTTCATTCATTATTAAC
SEQ ID NO: 12-atuL-H 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAATCCGCCTTTAACACCATCAGCAGGGC
CTCTTCGGCCAATTCTTCAAGGGTCAGGCTGCCCTGGGTACGGAACCAGGTAGTGGTCCAGGACA
ACGCGCCCGTCAGGAAACGCCGAGTAATAAACACGTCACCGCGGATATACCCGGCGGTCTTGGC
CTCACCCAATACCTGCAACCAGATATCTTCATAAACGTCGCGCAAGGCCAGCACCTGGGCCTGGC
CCTCGGCCGACAGCGAACGCCATTCGTAGACCAGCACCGCCATGGCCTCGCCGCTGCCGCCCATG
ATCGACTGCAACTCGCAGCGAATCAGCGCCAGCACACGCTCGCGTACAGTGCTCGCCTCTTCCAG
CGCGGCGCGCATCATTGCGGTGTTGTAGTGGATGGTTTCTTCCATCACCGCCCGCAGAATCTCGTC
CTTGCTCTTGAAATGATGAAAGATGCTGCCCGACTGTATGCCCACGGCGCTGGCAAGGTCACGCA
CGGTAGTGCGCTCAAAACCCTTGTTGCGAAACAGGTGAGCCGCGGTTTGCAGCAATTTGCCCCGG
GCGCTCTCGGGGTCGGTCAATCGGCCACCGGCGACCATGGCGCGCATCACCCCCAGGGCTTTGTG
CTCATCCATATGGGGTGGCCCCTGATTGTTGTTGTTTGATCAGCTTGGATAATTGGTAATACCAAT
TTACAATCGCTGTGGGCTAGATTAAGAGCCTTGGCAGGGGTGTGTCAATTTGCCGCTCGTCAAAC
TTTGGTCTGAGAGTGGCTGTCGAGTGTTCTGAACGGGTAAATCGGGACCAAAACGAAAAAAGGC
CCCCCTTTCGGGAGGCCTCTTTTCTGGAATTTGGTACCGAGGAGAAATCCGTCCTCCAGGGGCTG
GTGTCGAGACTACCAAGCAAGCGCTTGGTTTGTAAACTGTGCGCACAAGTTTTACCCAAGCGCTT
GCTTGGGTGGCGGCCACGGCCTAAATTGCCGCCCAAGATGTCAGCACAAGTGAAGGAGAGCAGC
ATGACTAAAAAAATTTCATTCATTATTAAC
SEQ ID NO: 13-marR 1.0
AATAGGCGTATCACGAGGCCCTTTCGTCTTCACCTCGAGGAGCAAGCCCCTCCCACATTCGCCCG
GCGGTGTGGCCGCTGAATCGCTCCATCCATCGAGAAATTGTTTCAAAACTTGACGAAGACTCGCC
CCTCCACCATATTAATAGTTAGCAAACTAACTGTATGCGAACTATCCAGTGTCCCAATCCCTCGAA
CAACTTCAGTCTAGTTGCGGCCGCAAAATGGATGGCAAATATGACTAAA
SEQ ID NO: 14-marR 2.0
TCACGAGGCCCTTTCGTCTTCACCTCGAGGGTAAATGCATCTGTGGCTGGCCCATCGCAATCGGG
AGCAAGCCCCTCCCACATTCGCCCGGCGGTGTGGCCGCTGAATCGCTCCATCCATCGAGAAATTG
TTTCAAAACTTGACGAAGACTCGCCCCTCCACCATATTAATAGTTAGCAAACTAACTGTATGCGAA
CTATCCAGTGTCCCAATCCCTCGAACAACTTCAGATGACTAAAAAAATTTCATTCATTATTAAC
SEQ ID NO: 15-marR-L 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAAGGCGCCACAGCCTCGGCCGCTTCGAA
GGCGCGCAGCACGCGCAGTGCGGCTTCCAGGTCGGTGGGCGCGATATCCACAAGGACTTCCTTG
CGCAGGCGAACCAGTTGGGCTTCTACCGCCTGCACCAGTTCACGGCCGCGCTCGGTGAGGCTCAG
GGCCTTGGCGCGACGGTCGTGGATGTCTTCGGTGCGGCATACGTAGCCGTCGCCGCACAGCCGA
TCGAGCAGGCGCACCAGTGACGGGCTCTCCATGCCGGAGGCCTGGGCTACTTTGACCTGGTGCA
CACCGTCACCCAGGCGGCCGATCATCAACAACGGCACGGCGCAGGCTTCGGAGATGCCATAGCT
CACCAGCGTGGTCTGGCAGATCTTGCGCCAGTTACGGGCGCCCACCACCATGCTGCTGCTCAGGT
TCATGGCGCAGATGCTCCAGGCAGTGCGAGAGAGAAACACAAGGCAACGGTTATCCCGGGACAT
CGATTTTTCGGCAGTCGACGGACGGGTGTTATAAAGACACCCCGAAAATTCTTGAGCCCGCCGAA
GCGGTGATCAGGCGCTGAAGTGGCCTGTTTCAAGGGTTTTTGGACCAAAACGAAAAAAGGCCCC
CCTTTCGGGAGGCCTCTTTTCTGGAATTTGGTACCGAGGTAAATGCATCTGTGGCTGGCCCATCGC
AATCGGGAGCAAGCCCCTCCCACATTCGCCCGGCGGTGTGGCCGCTGAATCGCTCCATCCATCGA
GAAATTGTTTCAAAACTTGACGAAGACTCGCCCCTCCACCATATTAATAGTTAGCAAACTAACTGT
ATGCGAACTATCCAGTGTCCCAATCCCTCGAACAACTTCAGATGACTAAAAAAATTTCATTCATTA
TTAAC
SEQ ID NO: 16-marR-M 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAAGGCGCCACAGCCTCGGCCGCTTCGAA
GGCGCGCAGCACGCGCAGTGCGGCTTCCAGGTCGGTGGGCGCGATATCCACAAGGACTTCCTTG
CGCAGGCGAACCAGTTGGGCTTCTACCGCCTGCACCAGTTCACGGCCGCGCTCGGTGAGGCTCAG
GGCCTTGGCGCGACGGTCGTGGATGTCTTCGGTGCGGCATACGTAGCCGTCGCCGCACAGCCGA
TCGAGCAGGCGCACCAGTGACGGGCTCTCCATGCCGGAGGCCTGGGCTACTTTGACCTGGTGCA
CACCGTCACCCAGGCGGCCGATCATCAACAACGGCACGGCGCAGGCTTCGGAGATGCCATAGCT
CACCAGCGTGGTCTGGCAGATCTTGCGCCAGTTACGGGCGCCCACCACCATGCTGCTGCTCAGGT
TCATATCGTGAGCTTCCTTACACACGGAAAGCCCTAACGATAGATGGTCCTGAGCGAAACGCATC
AGGGTGTCGGCGTCTCGGGACCCGACCTAGCTTTCCTGTAGGAACTATCTCTTAGCGACGTAGGG
GGAAGTACAATTCAGAACAATTGTTCAGAATTGTGCGATCGGACCAAAACGAAAAAAGGCCCCCC
TTTCGGGAGGCCTCTTTTCTGGAATTTGGTACCGAGGTAAATGCATCTGTGGCTGGCCCATCGCA
ATCGGGAGCAAGCCCCTCCCACATTCGCCCGGCGGTGTGGCCGCTGAATCGCTCCATCCATCGAG
AAATTGTTTCAAAACTTGACGAAGACTCGCCCCTCCACCATATTAATAGTTAGCAAACTAACTGTA
TGCGAACTATCCAGTGTCCCAATCCCTCGAACAACTTCAGATGACTAAAAAAATTTCATTCATTAT
TAAC
SEQ ID NO: 17-marR-H 3.0
TCACGAGGCCCTTTCGTCTTCACCTCGAGGGGGAGACCAGAAACAAAAAAAGGGGAGCGGTTTC
CCGCTCCCCTTCAATAATTGGTTAATAGCGTGCACCTCAAGGCGCCACAGCCTCGGCCGCTTCGAA
GGCGCGCAGCACGCGCAGTGCGGCTTCCAGGTCGGTGGGCGCGATATCCACAAGGACTTCCTTG
CGCAGGCGAACCAGTTGGGCTTCTACCGCCTGCACCAGTTCACGGCCGCGCTCGGTGAGGCTCAG
GGCCTTGGCGCGACGGTCGTGGATGTCTTCGGTGCGGCATACGTAGCCGTCGCCGCACAGCCGA
TCGAGCAGGCGCACCAGTGACGGGCTCTCCATGCCGGAGGCCTGGGCTACTTTGACCTGGTGCA
CACCGTCACCCAGGCGGCCGATCATCAACAACGGCACGGCGCAGGCTTCGGAGATGCCATAGCT
CACCAGCGTGGTCTGGCAGATCTTGCGCCAGTTACGGGCGCCCACCACCATGCTGCTGCTCAGGT
TCATATGGGGTGGCCCCTGATTGTTGTTGTTTGATCAGCTTGGATAATTGGTAATACCAATTTACA
ATCGCTGTGGGCTAGATTAAGAGCCTTGGCAGGGGTGTGTCAATTTGCCGCTCGTCAAACTTTGG
TCTGAGAGTGGCTGTCGAGTGTTCTGAACGGGTAAATCGGGACCAAAACGAAAAAAGGCCCCCC
TTTCGGGAGGCCTCTTTTCTGGAATTTGGTACCGAGGTAAATGCATCTGTGGCTGGCCCATCGCA
ATCGGGAGCAAGCCCCTCCCACATTCGCCCGGCGGTGTGGCCGCTGAATCGCTCCATCCATCGAG
AAATTGTTTCAAAACTTGACGAAGACTCGCCCCTCCACCATATTAATAGTTAGCAAACTAACTGTA
TGCGAACTATCCAGTGTCCCAATCCCTCGAACAACTTCAGATGACTAAAAAAATTTCATTCATTAT
TAAC
SEQ ID NO: 18-3680 1.0
AATAGGCGTATCACGAGGCCCTTTCGTCTTCACCTCGAGGCACCCCGGTGCAAAACCGCGTGCTT
GAGAGGCTCGATCTAGAGCCTTTGCAGGGAACAATCAATTCTGTTGATTATTACCATTGTGTTTAT
GAACTTTTATATCGACTTATCAAGAGTAGCATTAGCCCTGTACCCACTTTATCGCCCTCCCAAGGA
GCAACCATCTTCTAGTTGCGGCCGCAAAATGGATGGCAAATATGACTAA
SEQ ID NO: 19-3680 2.0
TCACGAGGCCCTTTCGTCTTCACCTCGAGGTGCACAGGGCTCCTGAATACGACTGGCTGGGCAAC
AGCCTTTGACACAGCTTATAGAACTACGACTGGCAAAGTGCCGGAGGGTTTAACCCAACGGCGG
ATTTAGCCGAAAAAAACGATTTTCCTGATCAAGCAGACAGATTTCATCATCGACAGCGCACCATTG
TTACCCCGGTTCGTGTCGAAAATACCCACCCCGGTGCAAAACCGCGTGCTTGAGAGGCTCGATCT
AGAGCCTTTGCAGGGAACAATCAATTCTGTTGATTATTACCATTGTGTTTATGAACTTTTATATCGA
CTTATCAAGAGTAGCATTAGCCCTGTACCCACTTTATCGCCCTCCCAAGGAGCAACCATCATGACT
AAAAAAATTTCATTCATTATTAAC
SEQ ID NO: 20-20745 2.0
TCACGAGGCCCTTTCGTCTTCACCTCGAGGCTTGGCGCACTCGTTCGCAGGCCGCAAGCCGA
TCGCCCTCTAACGCCCATTGCAGCCCCAATGTGGGAGCTGGCTTGCCTGCGATAGCATCACCACG
GTGTAACTGAAAAACCGAGGCGCTTGCATCGCGGGCAAGCCCGGCTCCCACATTGGTTTTGTGGT
GTCGGTGAAAATCGTGTACAGCTTGAAAACCAAGCAAGCGCTTGGTAAGTTCGCTCCATCTCGCC
ATGGAGCTTGCCGATGACTAAAAAAATTTCATTCATTATTAAC

In some embodiments, the genetic circuit may be moved to the genome from a plasmid. In some embodiments, the invention comprises an expression vector comprising the above genetic circuit.

In some embodiments, the invention comprises a host cell transformed with the above genetic circuit.

The bacterial biosensors may be used to indicate the presence of NAs in an environment during various methods. In some embodiments, the environment comprises water, soil, or sediment. In some embodiments, the water is sourced from the ground, soil, river, lake, stream, pond, tailings pond, wastewater, or OSPW. In some embodiments, the water is OSPW. Extraction of NAs from the environment (for example, water) is unnecessary and environmental samples (for example, water) can be collected and sent to a laboratory for testing directly with minimal treatment, which may or may not include sterilization (UV, filtration) or concentration (evaporation). Upon detection of NAs within minutes, the bacterial biosensors induce the expression of one or more key genes which are fused as a transcriptional reporter. The genetic circuit facilitates the production of a color change, fluorescence signal, or bioluminescence, and the amount of color, electrochemical or light is directly proportional to the concentration of NAs in the sample, thereby acting as a semi-quantitative indicator of a range of total NA concentrations between 1-120 mg/L, with an accuracy range of plus/minus 20 mg/L, based on comparisons to a standard curve. In some embodiments, the concentration of NAs ranges between 1-40 mg/L. In some embodiments, the concentration of NAs ranges between 20-60 mg/L. In some embodiments, the concentration of NAs ranges between 60-100 mg/L.

In some embodiments, the invention comprises a method of detecting the presence of NAs in an environment comprising: providing a sample from the environment suspected of containing NAs; and contacting the sample with a bacterial biosensor; wherein a detectable signal is induced in the bacterial biosensor, the detectable signal being indicative of NAs in the environment.

In some embodiments, the invention comprises a method for detecting the presence of NAs in an environment comprising: contacting a sample from the environment suspected of containing NAs with a bacterial biosensor; and detecting color or light emitted by a gene product that is induced by the presence of NAs, the intensity of the color or light being indicative of the concentration of NAs in the environment.

In some embodiments, the environment comprises water from the ground, soil, river, lake, stream, pond, tailings pond, wastewater, or oil sands process-affected water. In some embodiments, the water comprises oil sands process-affected water. In some embodiments, the naphthenic acids in the oil sands process-affected water range in concentration from about 20 mg/L to about 120 mg/L. In some embodiments, the detectable signal comprises color or light emitted by locate, beta-galactosidase, or green fluorescent protein.

In some embodiments, the invention comprises a method for monitoring degradation of NAs by a bacterial strain comprising: growing a bacterial strain in the presence of a known amount of NAs; and contacting the bacterial strain with a bacterial biosensor; wherein a detectable signal is induced in the biosensor, the detectable signal being indicative of NAs degraded by the bacterial strain. In some embodiments, the detectable signal comprises color, electrochemical, or light emitted by luxCDABE, beta-galactosidase, or green fluorescent protein. Candidate bioremediation strains are grown with known amounts of NAs, and then the biosensor is added in a second, co-culture step, whereby the biosensor will respond quantitatively to the amount of NAs remaining after degradation. This is compared to positive controls with no bioremediation capacity to determine the percentage degradation. This method would support the rapid identification of NA remediation strains without extraction of NAs nor analytical chemistry methods for quantitation.

During the development of the invention, a bacterial transcriptomics approach was applied to identify multiple bacterial promoters that are highly expressed in the presence of NAs. The NA-induced genes were identified within a bacterial isolate which was isolated from OSPW obtained from the Athabasca oil sands region in Alberta, Canada. The primary genes of interest are likely needed to use NAs as a carbon source or were involved in efflux of toxic NA compounds within the mixture. Genetic circuits were constructed using synthetic biology, where the upstream promoter regions from biologically meaningful NA-induced genes were identified and extracted using bioinformatics, and then cloned upstream of gene expression reporters in plasmids. The resulting genetic circuits were expressed on plasmids that are introduced back into the oil sands bacterial isolate. Several rounds of optimization were performed in attempt to improve the expression and sensitivity of the NA biosensors. Routine microbiology and gene expression assays using 96 well plates were performed to grow the biosensor strains in the presence of environmental water samples, and the light output from the biosensor was used as a proxy to indicate the concentration of naphthenic acids in the water. The biosensors may be adapted for use in field testing using handheld luminometers, or can be redesigned with other reporters and other field ready detectors. The genes involved in the biosensor have a strong biological rationale and relevance for responding to NA. In addition, the mechanism of NA sensing was identified by demonstrating that the transcriptional repressors employed in the biosensor bind and therefore sense NAs, which further validates the biological foundation for the bacterial sensing and response processes to NAs.

As described in the Examples, multiple and distinct genetic circuits, each with a unique promoter, have been constructed as a panel of biosensors that permit the identification of a wide range of NAs. The bacterial biosensors detect and respond to specific NAs, with low limits of detection, including the concentrations of NAs in tailings ponds. The genetic circuits have been optimized for strong gene expression and high signal responses above the background control conditions.

Embodiments of the present invention are described in the following Examples, which are set forth to aid in the understanding of the invention and should not be construed to limit in any way the scope of the invention as defined in the claims which follow thereafter.

### Example 1—Bacterial Biosensor Construction

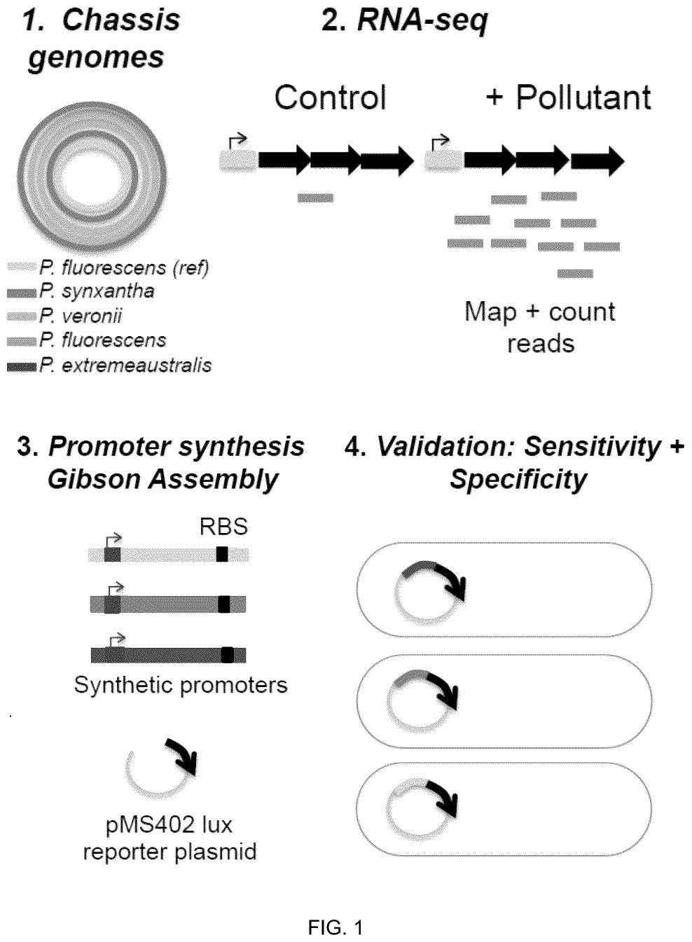

As shown in FIG. 1, a pipeline (steps 1-4) was developed using bacterial genomics and synthetic biology to construct naphthenic acid biosensor plasmids and strains for use in detecting a target environmental pollutant. In the first step, Pseudomonas isolates were cultivated directly from OSPW onto Pseudomonas isolation agar. Suitable media for growing the bacterial biosensors include, but are not limited to M9 or BM2 defined, minimal media with succinate as a carbon source. The genomes of OSPW isolates were sequenced by obtaining and combining short sequencing reads (Illumina, San Diego, CA) and long sequencing reads (Oxford Nanopore Technologies, Oxford, UK). A complete genome of Pseudomonas strain OST1909 has been reported, and may be a novel species according to digital DNA-DNA hybridization analysis (Shideler et al., 2021).

In the second step, bacterial genes which are highly induced by the presence of naphthenic acids were identified. RNA-seq was used to determine the transcriptome of Pseudomonas OST1909 grown in the presence of three different naphthenic acid samples: 1) commercially available NAs consisting of mainly acyclic NAs, 2) a custom mixture composed of nine individual compounds and 3) OSPW-extracted NAs. OSPW and NA samples from Alberta were provided by John Headley (Environment and Climate Change Canada, National Hydrology Research Centre, Saskatchewan, Canada) (Table 2). The genes/promoters in front of the NA-induced genes were prioritized for biosensor construction if their predicted gene function was involved in bioremediation, efflux, transport, or transcription regulation.

TABLE 2
Naphthenic Acids Sample Types and Sources

| | | | | |
|---|---|---|---|---|
| Compound | Abbreviation | Supplier | Catalogue # | CAS # |

Custom Mix Composition

| | | | | |
|---|---|---|---|---|
| 5,6,7,8-Tetrahydro-2- | THNA | Sigma Aldrich | 67856-5G | 1131-63-1 |
| naphthoic acid | | | | |
| 1-Adamantanecarboxylic acid | 1-ACA | Sigma Aldrich | 106399-25G | 828-51-3 |
| Cyclohexane acetic acid | CHAA | SAFC | W234702-25G | 5292-21-7 |
| 3-Phenylpropionic acid | 3PPA | Sigma Aldrich | W288918-100G-K | 501-52-0 |
| Cyclohexane pentanoic acid | CHPA | Sigma Aldrich | 331562-5G | 5962-88-9 |
| Cyclopentane carboxylic acid | CPCA | Sigma Aldrich | C112003-5G | 3400-45-1 |
| Hexanoic Acid | HA | Sigma Aldrich | 153745-100G | 142-62-1 |
| Decanoic Acid | DA | Sigma Aldrich | C1875-100G | 334-48-5 |
| a,4-Dimethylphenylacetic acid | DMPAA | Sigma Aldrich | 522449-5G | 938-94-3 |

Sigma Commercial
Naphthenic Acid

| | | | | |
|---|---|---|---|---|
| Naphthenic Acid | SIGNA | Sigma Aldrich | 70340-250ML | 1338-24-5 |

Environmental Naphthenic
Acid Samples

| | | | | |
|---|---|---|---|---|
| OSPW Acid Extractable | ECNA | John Headley | N/A | N/A |
| Organics (AEO) | | | | |

### Example 2—Promoter Prediction, Synthesis and Version 1.0 Cloning

In the third step, the upstream 165-400 bp from the differentially expressed genes was extracted for promoter analysis. The presence of sigma 70-like promoters and known transcription factor sites was confirmed using a bacterial promoter prediction program (“BPROM”) analysis. Sixty-two promoters (minimal length of about 165 bp) were synthesized using the BioXP™ DNA printer (Synthetic Genomics Inc., La Jolla, CA) upstream of genes induced by exposure to NAs. Table 3 lists all the NA-induced promoters/genes that were cloned as biosensor genetic circuits (version 1.0).

TABLE 3
Promoters used for biosensor construction version 1.0

| Locus Tag | Lab Name | Description |
|---|---|---|
| IH404_19875 | ECNA_14 | 3-hydroxy-3isohexenylglutaryl-CoA:acetate lyase |
| IH404_21810 | 9 × NA_1 | Magnesium and cobalt transport protein CorA |
| IH404_13000 | 9 × NA_10 | FIG00957600: hypothetical protein |
| IH404_17720 | 9 × NA_11 | Acetoacetyl-CoA synthetase |
| IH404_00740 | 9 × NA_12 | Phosphatidate cytidylyltransferase |
| IH404_21755 | 9 × NA_14 | Outer membrane protein romA |
| IH404_12850 | 9 × NA_15 | SOS-response repressor and protease LexA |
| IH404_18050 | 9 × NA_2 | Copper resistance protein |
| IH404_03680 | 9 × NA_3 | FIG00963947: hypothetical protein |
| IH404_19800 | 9 × NA_4 | FIG00953060: hypothetical protein |
| IH404_22720 | 9 × NA_5 | Dicarboxylate MFS transporter |
| IH404_06465 | 9 × NA_6 | 4-hydroxybenzoate transporter |
| IH404_07575 | 9 × NA_7 | Transcriptional regulator - TetR family |
| IH404_05955 | 9 × NA_8 | Medium-chain-fatty-acid-CoA ligase |
| IH404_25955 | 9 × NA_9 | Acyl-CoA dehydrogenase |
| IH404_13235 | ECNA_1 | Transcriptional regulator2C GntR family domain |
| IH404_15675 | ECNA_10 | Undecaprenyl-diphosphatase |
| IH404_20295 | ECNA_11 | FIG00960278: hypothetical protein |
| IH404_14720 | ECNA_13 | Probable acyl-CoA dehydrogenase |
| IH404_24395 | ECNA_12 | Pxn resistance; undecaprenyl phosphate-alpha-L-Ara4N |
| | | transferase |
| IH404_10545 | ECNA_15 | Quaternary ammonium compound-resistance protein SugE |
| IH404_11000 | ECNA_2 | Cytosine permease |
| IH404_12170 | ECNA_3 | Biotin synthase related domain containing protein |
| IH404_13065 | ECNA_4 | Transcriptional regulator2C GntR family |
| IH404_08160 | ECNA_5 | Transcriptional regulator2C LysR family |
| IH404_10425 | ECNA_6 | hypothetical protein |
| IH404_18430 | ECNA_7 | FIG074102: hypothetical protein |
| IH404_15830 | ECNA_8 | FIG00957990: hypothetical protein |
| IH404_12910 | SIGNA_1 | Cyclohexanone monooxygenase |
| IH404_21035 | SIGNA_10 | 2-methylcitrate synthase |
| IH404_17590 | SIGNA_12 | Permease of the drug/metabolite transporter (DMT) |
| | | superfamily |
| IH404_21045 | SIGNA_13 | Methylisocitrate lyase |
| IH404_19135 | SIGNA_14 | Succinate-semialdehyde dehydrogenase |
| IH404_18550 | SIGNA_15 | Putative exported protein precursor |
| IH404_16165 | SIGNA_16 | Histone acetyltransferase |
| IH404_11365 | SIGNA_17 | CDP-diacylglycerol--glycerol-3-phosphate 3- |
| | | phosphatidyltransferase |
| IH404_08355 | SIGNA_18 | RND efflux system2C membrane fusion protein CmeA |
| IH404_23675 | SIGNA_19 | Sulfate permease |
| IH404_16675 | SIGNA_2 | Major facilitator superfamily |
| IH404_23035 | SIGNA_20 | Putative preQ transporter |
| IH404_08730 | SIGNA_21 | Metal-dependent hydrolases of the beta-lactamase |
| | | superfamily |
| IH404_27030 | SIGNA_22 | hypothetical protein |
| IH404_09140 | SIGNA_23 | Enoyl-CoA hydratase |
| IH404_27030 | SIGNA_24 | Lead, cadmium, zinc and mercury transporting ATPase |
| IH404_19875 | SIGNA_29 | Citronellyl-CoA dehydrogenase |
| IH404_04355 | SIGNA_3 | Heavy metal sensor histidine kinase |
| IH404_23630 | SIGNA_30 | Alkylphosphonate utilization operon protein PhnA |
| IH404_16920 | SIGNA_31 | Multidrug resistance transporter Bcr/CflA family |
| IH404_12850 | SIGNA_32 | SOS-response repressor and protease LexA |
| IH404_01620 | SIGNA_4 | MarR transcriptional regulator; fusaric acid resistance |
| IH404_12915 | SIGNA_5 | Beta-ketoadipate enol-lactone hydrolase |
| IH404_19870 | SIGNA_6 | Acyclic terpenes utilization regulator AtuR -TetR family |
| IH404_15770 | SIGNA_7 | Putative NADPH-quinone reductase |
| IH404_17665 | SIGNA_8 | Outer membrane component of tripartite multidrug |
| | | resistance system |
| IH404_21030 | SIGNA_9 | 2-methylaconitate cis-trans isomerase |

*Promoters beginning with “ECNA” were from genes induced by naphthenic acids provided by Environment Canada; promoters beginning with “SIGNA” were from genes induced by naphthenic acids provided by commercial vendor Sigma Aldrich; promoters beginning with “9 × NA” were from genes induced by naphthenic acids present in a custom mix of 9 individual naphthenic acid compounds.

The promoters were designed with 40 bp linkers that matched the overhangs generated by treating the lux-reporter plasmid with a single restriction enzyme and a 5′ exonuclease. Gibson Assembly™ was performed using a BioXP™ system (Codex DNA, Inc., San Diego, CA) to clone the promoters of interest upstream of the luxCDABE reporter plasmid pMS402. In the version 1.0 constructs, the pMS402 plasmid was not altered and new promoters were cloned with their own ribosome binding site (“RBS”) and also included the vector RBS directly upstream of the luxC start site. PCR and select DNA sequencing analysis confirmed that promoters were successfully synthesized and cloned. In the fourth step, all plasmid-encoded genetic circuits were introduced back into Pseudomonas OST1909 for validation.

### Example 3—Version 2.0 Cloning

For further optimization, a subset of promoters was cloned in the version 2.0 strategy where the 30 bp linkers were re-positioned such that the final constructs have the vector encoded RBS deleted (see Sequence Listing for 30 bp linker sequences SED ID NOS: 2, 8, 14, 19, 20). This optimization of RBS configuration permitted only the native Pseudomonas OST1909 RBS and promoter with the correct spacing in front of the luxC start codon, which resulted in much higher overall expression levels and the NA-inducibility was retained (data not shown). The 2.0 constructs had similar NA responses or were less sensitive to naphthenic acids compared to version 1.0 constructs. The most sensitive biosensor constructs were defined as those with the highest fold change values in response to NA.

### Example 4—Results and Discussion

The version 1.0 constructs contained promoter sequences only, and the 2.0 panel of NA-responsive biosensors focused on three main promoters: atuA, marR and a hypothetical gene IH404_03680 (SEQ ID NOS: 1-20).

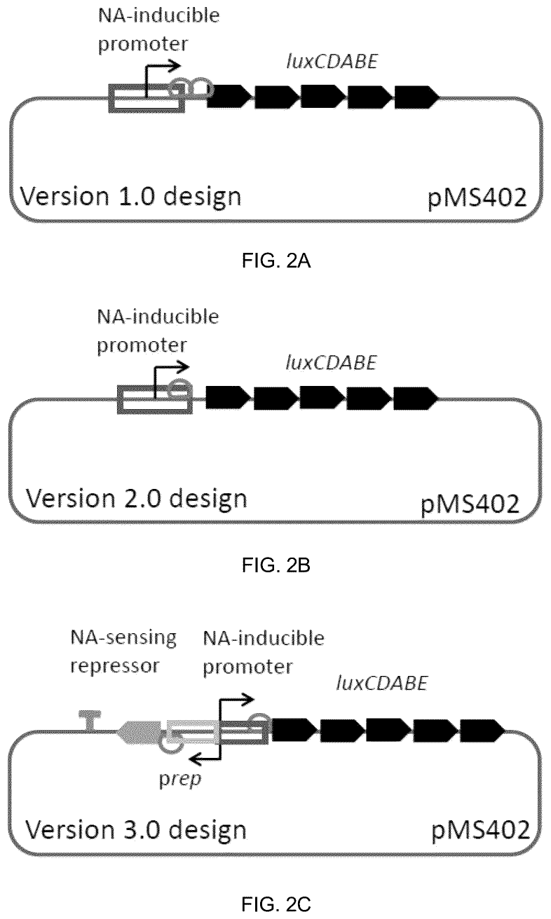

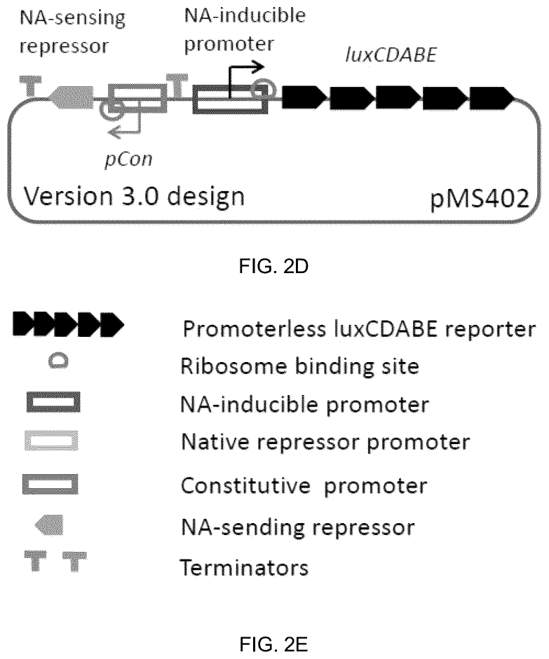

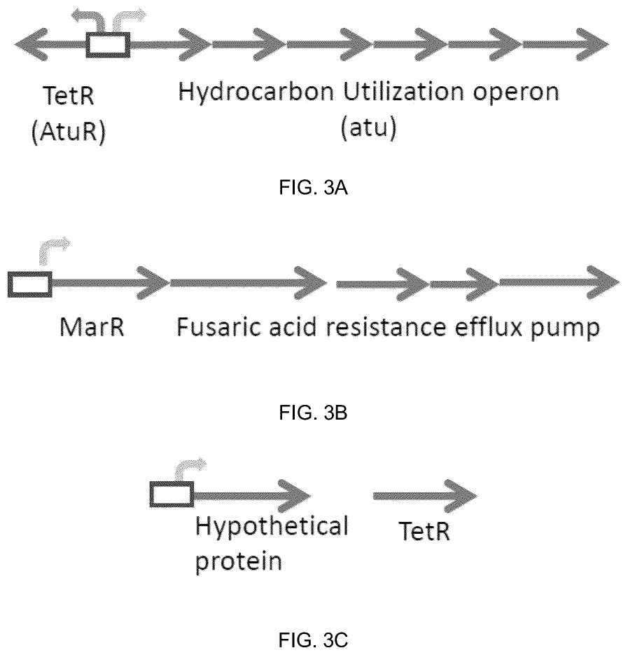

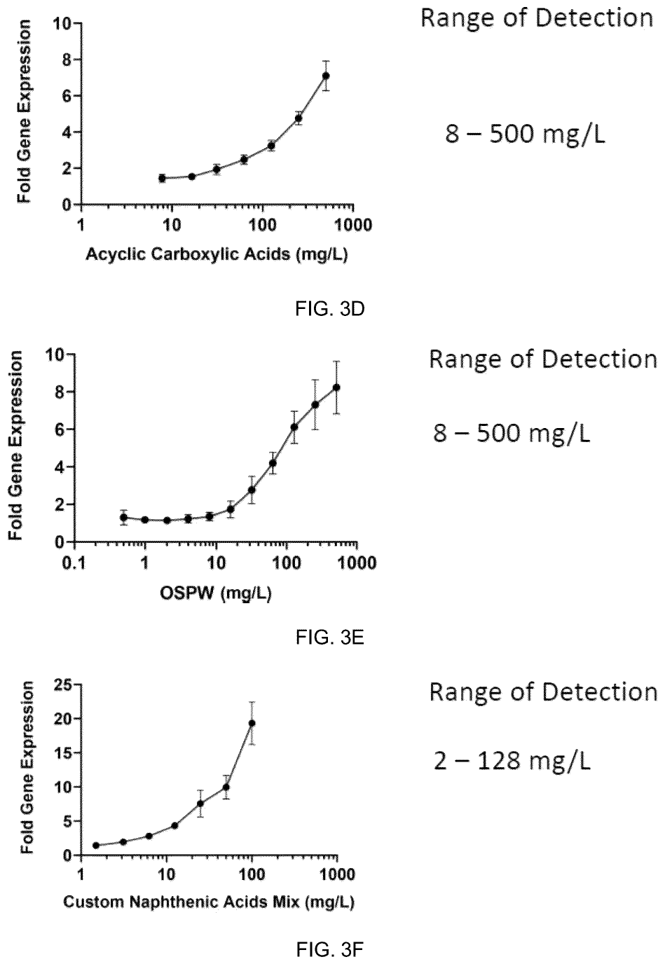

FIGS. 3A-F show the genetic organization of promoters that were synthesized and used in biosensor constructs for the detection of NAs (FIGS. 3A-C), and the linear, dose-dependent increase in gene expression by the biosensor in response to increasing concentrations of different mixtures of NAs (FIGS. 3D-F). The range of NA detection is 8-500 mg/L of acyclic NAs (FIG. 3D), 16-500 mg/L of OSPW (FIG. 3E) and 2->128 mg/L of a simple custom mixture of nine compounds (FIG. 3F). The dose-dependent gene expression responses allow these biosensors to provide a semi-quantitative indication of the concentration of NA in a sample.

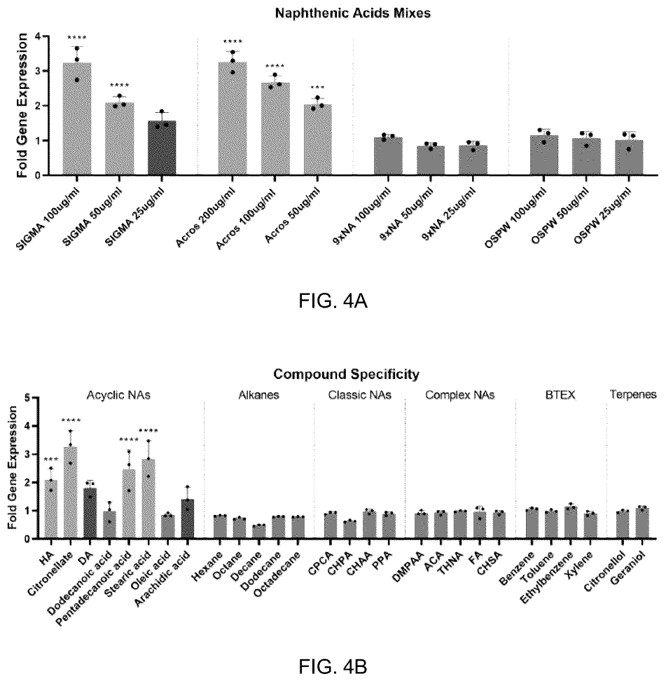

FIGS. 4A-B show detection by the atuA-L biosensor of acyclic NA mixes and individual NA compounds. FIG. 4A is a graph showing the fold gene expression values of four different NA mixes with decreasing concentrations. “SIGMA” and “Acros” indicate two sources of primarily acyclic NA mixtures. A one-way ANOVA found that there was a statistically significant difference between treatment groups (F(12,26)=51.43, p<0.0001). FIG. 4B is a graph showing the induction (fold gene induction) values of 28 different NAs at a concentration of 50 mg/L. A one-way ANOVA revealed a statistically significant difference between treatment groups (F(28,58)=20.96, p<0.0001). A post hoc Tukey HSD test was conducted with each compound and mixture to test significant relationships between each group (α=0.05, n=3). Treatment groups with significant results compared to the control are indicated with asterisks (*** indicate p values below 0.001, **** indicate p values below 0.0001). Nonsignificant groups displaying fold gene expression values above 1.5 are also shown.

Without being bound to any theory, the atu operon is likely a bioremediation operon and encodes enzymes that are required to use acyclic naphthenic acids as a carbon source (FIGS. 3A and D; FIGS. 4A-B). Note that these genes are required and induced by acyclic terpenes, such as citronellol and geraniol in Pseudomonas aeruginosa. However, acyclic terpenes (citronellol and geraniol) do not induce the corresponding operon in Pseudomonas OST1909, and the P. aeruginosa atu operon is not induced by naphthenic acids (data not shown). Since citronellic acid and other acyclic naphthenic acids induce expression of the atu genes in Pseudomonas OST1909, the atu genes are likely adapted for use in degrading related but unique carbon sources, namely acyclic naphthenic acids.

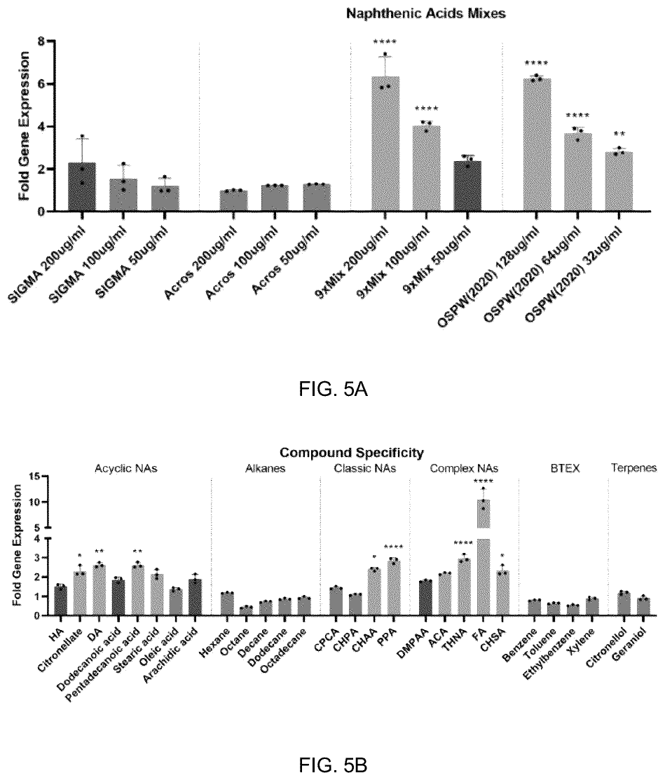

FIGS. 5A-B show detection by the marR-L biosensor of NA extracted from OSPW and individual NAs. FIG. 5A is a graph showing the fold gene expression values of four different NA mixtures with decreasing concentrations. Positive responses were detected against the 9xNA custom mixture and the NA extracted from OSPW. A one-way ANOVA found that there was a statistically significant difference between treatment groups (F(15,32)=34.83, p<0.0001). FIG. 5B is a graph showing the fold gene expression values of 28 different NAs at a concentration of 50 μg/mL. A one-way ANOVA revealed a statistically significant difference between treatment groups (F(28,58)=66.35, p<0.0001). A post hoc Tukey HSD test was conducted with each compound and mixture to test significant relationships between each group (α=0.05, n=3). Treatment groups with significant results compared to the control are indicated with asterisks (* indicate p values below 0.05, ** indicate p values below 0.01, indicate p values below 0.0001). Nonsignificant groups displaying fold gene expression values above 1.5 are also shown.

Fusaric acid is an antibiotic produced by fungi and also a naphthenic acid compound that contains a nitrogen-containing ring. Without being bound by any theory, the marR-efflux pump operon is likely needed to pump out fusaric acid, or similar NAs that are present with OSPW-extracted NAs (FIGS. 3B and E; FIGS. 5A-B). These data suggest that while some NA compounds are used as carbon sources (acyclic terpenes), others are toxic and need to be pumped out of the cell.

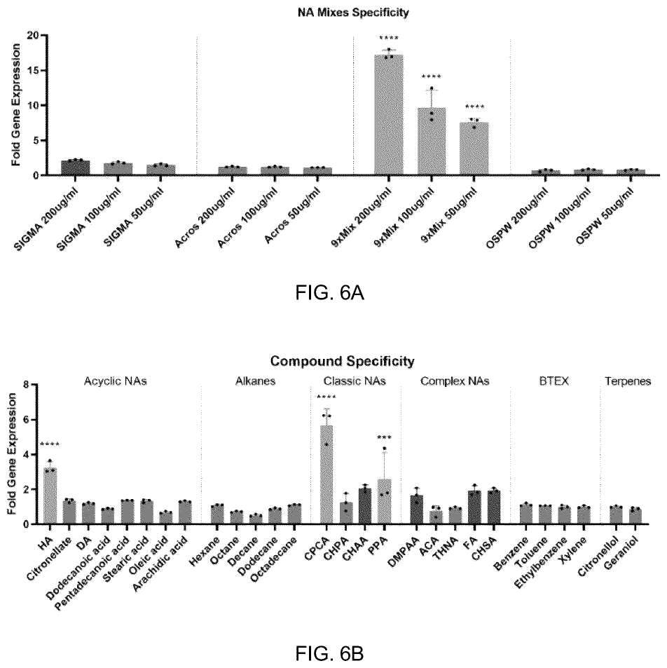

FIGS. 6A-B are graphs showing detection by the p3680 biosensor of the custom 9xNA mix and individual NA compounds. FIG. 6A is a graph showing the fold gene expression values of four different NA mixtures with decreasing concentrations. The 9xNA mix is composed of nine individual commercially available NA compounds. A one-way ANOVA found that there was a statistically significant difference between treatment groups (F(12,26)=144.6, p<0.0001). FIG. 6B is a graph showing the fold gene expression values of 28 different NAs at a concentration of 50 mg/L. A one-way ANOVA revealed a statistically significant difference between treatment groups (F(28,58)=21.66, p<0.0001). A post hoc Tukey HSD test was conducted with each compound and mix to test significant relationships between each group (α=0.05, n=3). Treatment groups with significant results compared to the control are indicated with asterisks (*** indicate p values below 0.001, *** indicate p values below 0.0001). Nonsignificant groups displaying fold gene expression values above 2 are also shown.

The third promoter IH404_03680 from a hypothetical protein (FIGS. 3C and F; FIG. 6) is thus very responsive to a simple mixture of nine compounds and detects specific compounds within the mixture. Given the gene encodes a hypothetical protein, its function in responding to simple NAs is not yet fully understood.

Based on the above results, all biosensors were capable of detecting NAs at concentrations known to exist in tailings ponds (20-120 mg/L).

### Example 5—Optimization of Version 3.0 Biosensor Constructs to Include NA-Sensing Proteins Into the Design

All promoters of interest are regulated by, or adjacent to, known small molecule sensing transcriptional repressors. TetR and MarR were originally discovered as antibiotic sensing repressors that no longer bind their repressed promoters in the presence of antibiotics, thereby expressing antibiotic resistance genes. Without being bound to any theory, this may likely be the same mechanism of repressing target genes in the absence of NAs, but after sensing and binding to NAs within the cell, they no longer act as repressors and gene induction is observed. The inventors have found that purified transcriptional repressors bind the predicted binding sites in the promoters, and that binding of naphthenic acids releases DNA binding. This was demonstrated using electrophoretic mobility shift assays (EMSA) with purified His-tagged transcriptional repressor proteins, DNA, and with NAs (data not shown).

Other biosensor designs include the NA-inducible promoter controlling the lux reporter and the corresponding transcriptional repressor protein within the genetic circuit (FIGS. 2C-D). In this way, the plasmid-encoded sensors contain both the “sensing” element and the “regulatory” element, which may enable these plasmids to function as self-contained biosensors when expressed in additional bacterial hosts, such as lab strains of E. coli. Indeed, the inventors have found that some of these genetic circuits sense NAs when expressed in E. coli. Within these version 3.0 constructs, various promoters were tested to control the transcriptional repressor (SEQ ID NOS: 3, 4, 5, 6, 9, 10, 11, 12, 15, 16, 17). We included the native promoter and constitutive promoters from Pseudomonas OST1909 previously determined by our lab to have low, medium or high levels of expression. The promoter with “low” strength is located upstream of a predicted BCCT family transporter (locus tag IH404_26360), the promoter with “medium” strength expression is found upstream of an arnT gene with 4-amino-4-deoxy-L-arabinose transferase activity (locus tag IH404_18330), and the “high” strength promoter is found upstream of a lactate permease LctP family transporter (locus tag IH404_24210) (FIG. 2D). A further design possibility was to chromosomally delete the expression of the repressor proteins, and to therefore express the repressor solely from the plasmid, under the control of various promoters, which may optimize the responses further. This mutational strategy was unsuccessful, but may be pursued in future experiments, as it may result in more controlled levels of the repressor protein and therefore improved sensitivity in detecting NAs.

### Example 6—NA Biosensor Sensitivity

The biosensors were constructed and tested to determine their limits of sensitivity, which is defined as the lowest detectable concentration of NAs. The biosensors sense and respond to specific compounds that are detected within the mixture, often within minutes or hours, with low limits of detection, below and including the concentration range of NAs in tailings ponds.

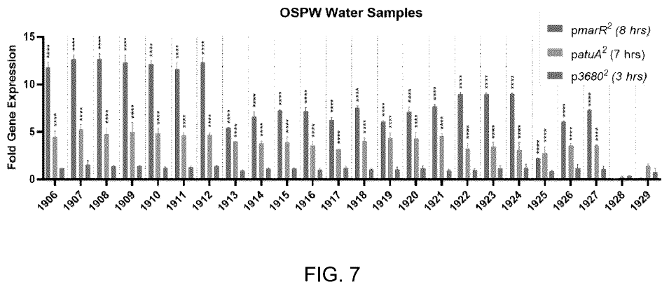

FIG. 7 shows the biosensor responses to naphthenic acids in OSPW samples. The fold gene expression values of marR-L, atuA-L, and the second generation p3680-based biosensor were taken at 8 hours, 7 hours, and 3 hours, respectively. Most samples from 1906 through 1929 induced significant biosensor responses upon exposure to 90 μl of an OSPW sample. A two-way ANOVA found a statistically significant difference between biosensor strains (F(2,150)=5867, p<0.0001) and NAFC samples (F(24,150)=194.0, p<0.0001). A post hoc Tukey HSD test was conducted with each water sample and extract to test significant relationships between each group (α=0.05, n=3). Treatment groups with significant results compared to the control are indicated with asterisks (*indicate p values below 0.05, ** indicate p values below 0.01, *** indicate p values below 0.001, **** indicate p values below 0.0001).

It should be apparent, however, to those skilled in the art that many more modifications besides those already described are possible without departing from the inventive concepts herein. The inventive subject matter, therefore, is not to be restricted except in the scope of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A biosensor for detecting the presence of naphthenic acids in an environment, comprising a modified Pseudomonas species carrying a plasmid or chromosomally encoding a genetic circuit comprising a reporter gene fused with a promoter responsive to the presence of naphthenic acids, the genetic circuit being expressed in the presence of naphthenic acids in the environment.

2. The biosensor of claim 1, wherein the modified Pseudomonas species is selected from the Pseudomonas fluorescens complex of species, or Pseudomonas species strain OST1909.

3. The biosensor of claim 1, wherein the reporter gene is selected from luxCDABE, beta-galactosidase, or green fluorescent protein.

4. The biosensor of claim 1, wherein the promoter is selected from the promoter IH404_19875, atuA, IH404_01620, marR, IH404_03680, or 3680.

5. The biosensor of claim 1, wherein the genetic circuit comprises an isolated nucleic acid sequence as set forth in any one of SEQ ID NOS: 1-20, or an isolated nucleic acid sequence having at least 80%, at least 85%, at least 90%, at least 95%, at least 96%, at least 97%, at least 98%, or at least 99% sequence identity to any one of SEQ ID NOS: 1-20.

6. The biosensor of claim 1, wherein the genetic circuit comprises a promoter responsive to the presence of naphthenic acids, and a transcriptional repressor protein controlling the promoter.

7. An isolated nucleic acid sequence as set forth in any one of SEQ ID NOS: 1-20, or an isolated nucleic acid sequence having at least 80%, at least 85%, at least 90%, at least 95%, at least 96%, at least 97%, at least 98%, or at least 99% sequence identity to any one of SEQ ID NOS: 1-20.

8. An expression vector or a host cell comprising the isolated nucleic acid of claim 7.

9. A method of detecting the presence of naphthenic acids in an environment comprising:
providing a sample from the environment suspected of containing naphthenic acids; and
contacting the sample with the biosensor of claim 1;
wherein a detectable signal is induced in the biosensor, the detectable signal being indicative of naphthenic acids in the environment.

10. The method of claim 9, wherein the environment comprises water from the ground, soil, river, lake, stream, pond, tailings pond, wastewater, or oil sands process-affected water.

11. The method of claim 10, wherein the water comprises oil sands process-affected water.

12. The method of claim 11, wherein naphthenic acids range in concentration from about 20 mg/L to about 120 mg/L.

13. The method of claim 9, wherein the detectable signal comprises color, electrochemical, or light emitted by luxCDABE, beta-galactosidase, or green fluorescent protein.

14. A method for detecting the presence of naphthenic acids in an environment comprising:
contacting a sample from the environment suspected of containing naphthenic acids with the biosensor of claim 1; and
detecting color, electrochemical or light emitted by a gene product that is induced by the presence of NAs, the intensity of the color, electrochemical, or light being indicative of the concentration of NAs in the environment.

15. The method of claim 14, wherein the environment comprises water from the ground, soil, river, lake, stream, pond, tailings pond, wastewater, or oil sands process-affected water.

16. The method of claim 15, wherein the water comprises oil sands process-affected water.

17. The method of claim 14, wherein the color, electrochemical, or light is emitted by luxCDABE, beta-galactosidase, or green fluorescent protein.

18. A method for monitoring degradation of naphthenic acids by a bacterial strain comprising:
growing a bacterial strain in the presence of a known amount of naphthenic acids; and
contacting the bacterial strain with the biosensor of claim 1;
wherein a detectable signal is induced in the biosensor, the detectable signal being indicative of naphthenic acids degraded by the bacterial strain.

19. The method of claim 18, wherein the detectable signal comprises color, electrochemical, or light emitted by luxCDABE, beta-galactosidase, or green fluorescent protein.
